(19) United States
(12) Patent Application Publication
(10) Pub. No.: US 2012/0245503 A1
(43) Pub. Date: Sep. 27, 2012

(54) DISPOSABLE WRIST SUPPORT

(76) Inventor: Michael DeVries, Porter Ranch, CA (US)

(21) Appl. No.: 13/419,309

(22) Filed: Mar. 13, 2012

Related U.S. Application Data
(60) Provisional application No. 61/454,796, filed on Mar. 21, 2011.

(51) Int. Cl. A61F 5/00

(52) U.S. Cl. 602/21

(57) ABSTRACT

A disposable wrist support includes a body formed of a flexible impervious elastomer material and configured to be worn about a wearer's wrist. At least one support device is attached to the body. The support device is configured to provide support to the wearer's wrist.

### RELATED APPLICATION INFORMATION

This patent claims priority from Provisional Patent Application No. 61/454,796, filed Mar. 21, 2011, entitled DISPOSABLE WRIST SUPPORT.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### BACKGROUND

1. Field

This disclosure relates to wrist support devices incorporated into or compatible with disposable sterile gloves.

2. Description of the Related Art

A variety of occupations require the use of disposable protective gloves. These include doctors, veterinarians, dentists and dental hygienists, tattoo artists, workers in clean-room manufacturing or research facilities, and any other occupations that expose a worker and/or a patient to hazardous chemicals or pose a risk of biological contamination. Many of these occupations also require fine repetitive hand motions that may cause carpal tunnel syndrome, repetitive stress injury, De Quervains syndrome, arthritis, tendonitis, cramping, fatigue, and other conditions that result in pain in the worker's wrist or hand. The pain caused by these and other conditions may be alleviated through the use of a support that limits the motions of the wearer's wrist. Such supports typically comprise a rigid or slightly flexible support member held to the wear's wrist using an arrangement of heavy fabric straps. However, such supports cannot be worn under disposable gloves, and wearing a support over a disposable glove leaves the support exposed to chemicals, biological fluids, or other contaminants.

### DESCRIPTION OF THE DRAWINGS

FIG. 1 is a plan view of a disposable wrist support.

FIG. 2 is another plan view of the disposable wrist support of FIG. 1.

FIG. 3 is a plan view of a wrist strap.

FIG. 4 is a perspective view of the disposable wrist support of FIG. 1 on a wearer's wrist.

FIG. 5 is another perspective view of the disposable wrist support of FIG. 1 on a wearer's wrist.

FIG. 6 is a plan view of another disposable wrist support.

FIG. 7 is another plan view of the disposable wrist support of FIG. 6.

FIG. 8 is a perspective view of a disposable wrist support showing interchangeable support members.

FIG. 9 is a perspective view of the disposable wrist support of FIG. 6 on a wearer's wrist.

FIG. 10 is another perspective view of the disposable wrist support of FIG. 6 on a wearer's wrist.

FIG. 11 is a perspective view of another disposable wrist support on a wearer's wrist.

FIG. 12 is 1 plan view of the disposable wrist support of FIG. 11.

FIG. 13 is another plan view of the disposable wrist support of FIG. 11.

FIG. 14 is a perspective view of an ambidextrous disposable wrist support.

FIG. 15 is another perspective view of the ambidextrous disposable wrist support of FIG. 14.

FIG. 16 is a perspective view of the ambidextrous disposable wrist support of FIG. 14 on a wearer's wrist.

FIG. 17 is a plan view of another disposable wrist support.

FIG. 18 is a perspective view of the disposable wrist support of FIG. 17 on a wearer's wrist.

FIG. 19 is another perspective view of the disposable wrist support of FIG. 17 on the wearer's wrist.

### DETAILED DESCRIPTION

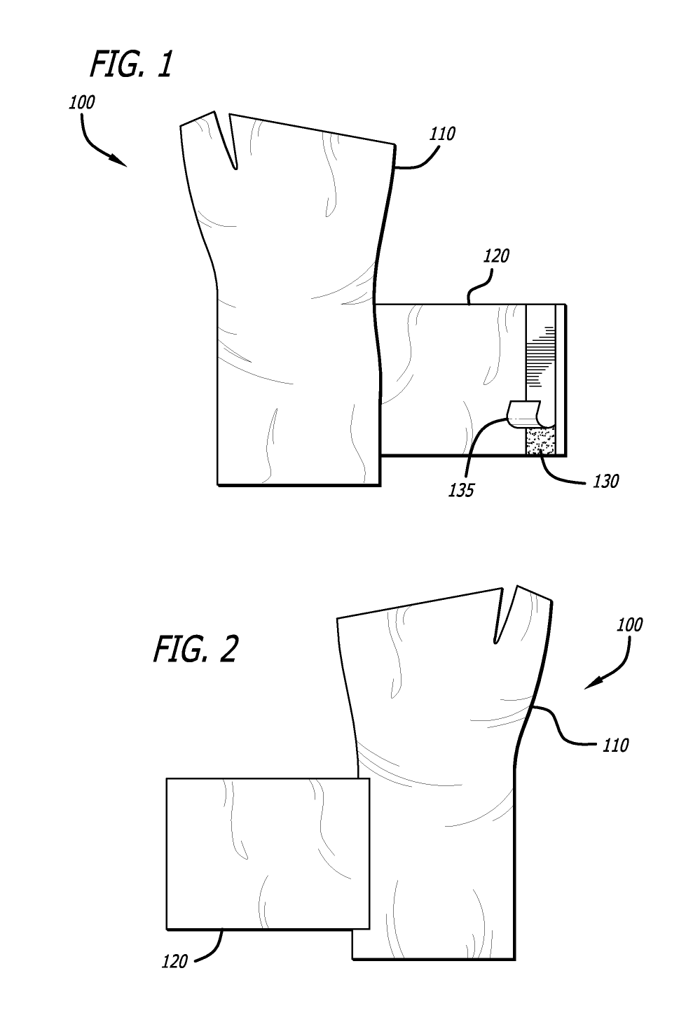

FIGS. 1, 2, 4, and 5 illustrate a disposable wrist support 100 that may be worn under or over a disposable protective glove. The disposable wrist support 100 includes a body 110 configured to be worn on a wearer's wrist and a support device in the form of a wrist strap 120. In this context, a “support device” is a device that provides support to a wearer's wrist. A support device may provide support by, for example, resisting motion of the wearer's wrist, limiting a range of motion of the wearer's wrist, and/or preventing motion of the wearer's wrist. The body 110 may be a fingerless glove as shown in FIG. 1 or may be a glove with full or partial fingers. When the body 110 is a fingerless glove, the body 110 may be worn over or under a conventional protective glove. One end of the wrist strap 120 may be fastened to the body 110. A second end of the wrist strap 120 may have an adhesive region 130 that may be exposed by removing a protective liner 135. A wearer may then fasten the wrist strap about their wrist to provide a desired level of support and compression, as shown in FIG. 4 and FIG. 5. The second end of the wrist strap 120 may have adhesive regions 130 and protective liners 135 on both an upper surface and a lower surface such that the wearer may wrap the wrist strap about their wrist in either direction.

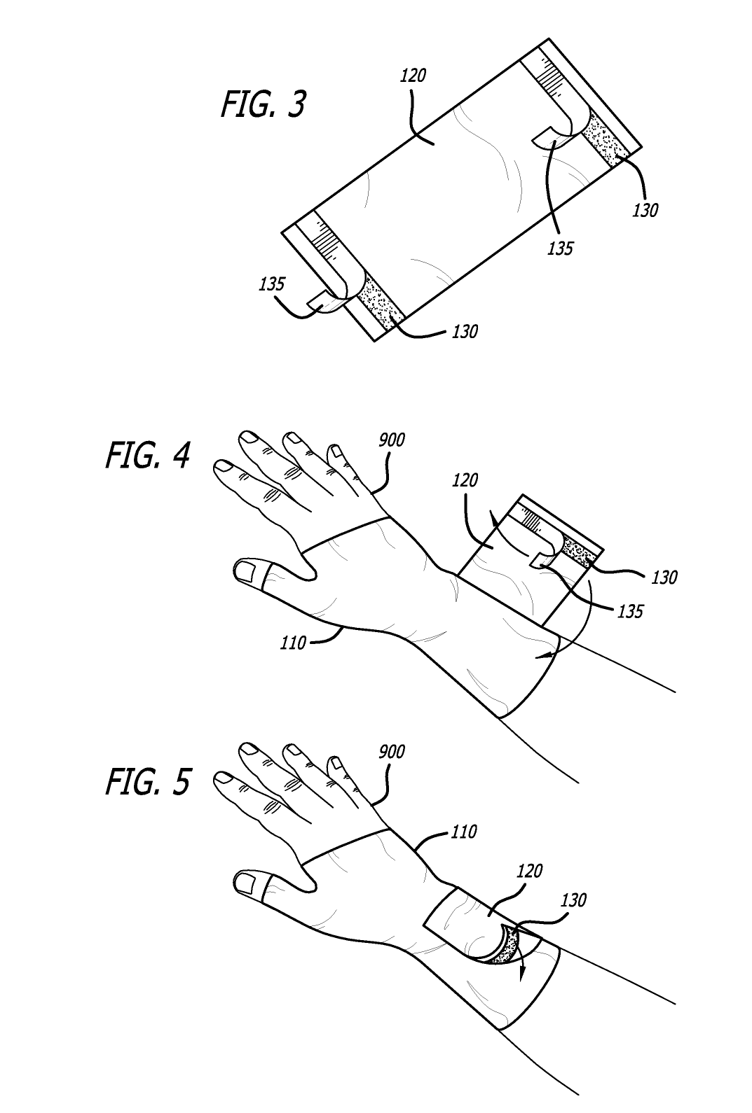

As shown in FIG. 3, the wrist strap 120 may be a separate component from the body 110 and have adhesive regions 130 and protective liners 135 at both ends. Providing adhesive regions at both ends of a wrist strap 120 may allow flexibility in positioning the wrist strap to provide the desired support for a wearer's wrist. A wrist strap with adhesive regions at both ends, as shown in FIG. 3, may be used to add support to a conventional sterile glove.

The body 110 and the wrist strap 120 may be made from a thin, flexible, stretchable elastomer material such as nitrile rubber, latex rubber, silicon elastomer, polyethylene, polyurethane, various co-polymers, and other elastomer materials. The material may be impervious. In this context, “impervious” means “incapable of being penetrated by one or more chemicals such as solvents, acids, and bases and/or biological contaminants such as microorganisms, blood, and other body fluids. The body 110 and the wrist strap 120 may be comprised of a single material layer, or two or more layers of the same or different materials. The type and thickness of the material may be selected to provide a desired combination of flexibility, support, and protection. The thickness of the material may be sufficiently thin to allow the disposable wrist support to be worn under conventional protective gloves. The material type and thickness of the body 110 and the wrist strap 120 may be the same or different. The body 110 may be provided with or without various inside surface treatments including aloe or powder.

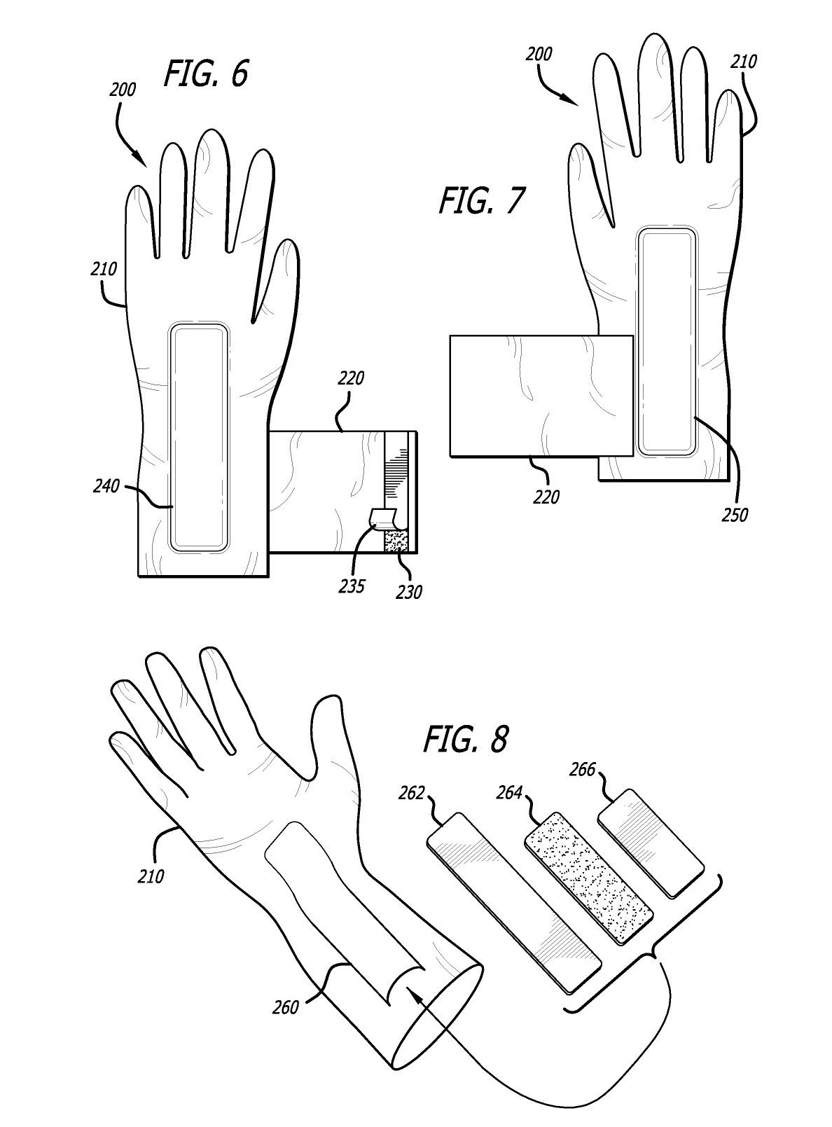

FIGS. 6, 7, 8, 9, and 10 show another disposable wrist support 200 that includes a body 210 and a support device including one or more of a wrist strap 220, an inner support member 240, and an outer support member 250. The body 210 may be a glove with full fingers, as shown, or may be a fingerless glove. One end of the wrist strap 220 may be fastened to the body 210. A second end of the wrist strap 220 may have an adhesive region 230 that may be exposed by removing a protective liner 235. The second end of the wrist strap 220 may have adhesive regions 230 and protective liners 235 on both an upper surface and a lower surface. A wearer may then wrap the wrist strap 220 about their wrist in either direction and fasten the wrist strap to provide a desired level of compression. An embodiment of the disposable wrist support 200 without a wrist strap is illustrated in FIG. 8.

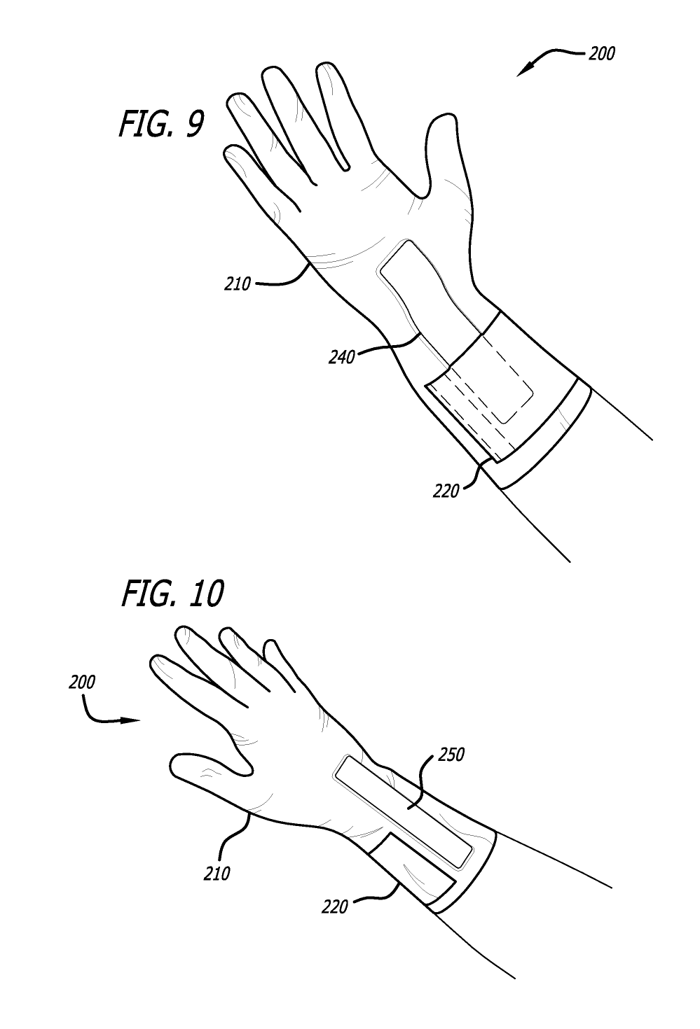

The inner support member 240 may be a rigid or slightly flexible member positioned proximate the inside of a wearer's wrist, as shown in FIG. 9, to prevent motion or to limit the range of motion of the wearer's wrist. The inner support member 240 may be fabricated from a rigid or slightly flexible material such as a metal, a plastic, or a composite material. The inner support member 240 may be deformable such that a wearer can form the support member into a preferred shape.

The inner support member 240 may be disposed within a pocket provided in the body 210. As shown in FIG. 6, the inner support member 240 may be sealed within a closed pocket formed, for example, by bonding or heat sealing two layers of elastomer material. Alternatively, as shown in FIG. 8, a removable inner support member 262, 264, 266 may be disposed in an open pocket 260, which is to say a pocket with an open end to allow a support member to be inserted or removed. In this case, two or more interchangeable removable inner support member 262, 264, 266 may be provided to allow a wearer to control the degree of support provided by the disposable wrist support 200. The interchangeable removable inner support members 262, 264, 266 may vary, for example, in length, stiffness, and/or material composition.

The outer support member 250 may be positioned proximate the outside of a wearer's wrist, as shown in FIG. 10. The material and characteristics of the outer support member 250 may be similar to those of the inner support member 240. The outer support member 250 may be disposed in a sealed pocket on the body 210, as shown in FIG. 7. Alternatively, a removable outer support member 250 may be disposed in an open pocket similar to that shown in FIG. 8. Interchangeable configurations of a removable outer support member may be provided to allow a wearer to control the degrees of support provided by the disposable wrist support 200.

The body 210 and the wrist strap 220 may be made from a thin, flexible, stretchable, impervious elastomer material as previously described. The weight or thickness of the material may be selected to provide a desired combination of flexibility, support, and protection. The material type and thickness of the body 210 and the wrist strap 220 may be the same or different. The body 210 may be provided with or without various inside surface treatments including aloe or powder.

FIG. 9 and FIG. 10 illustrate the disposable wrist support 200 worn by a wearer.

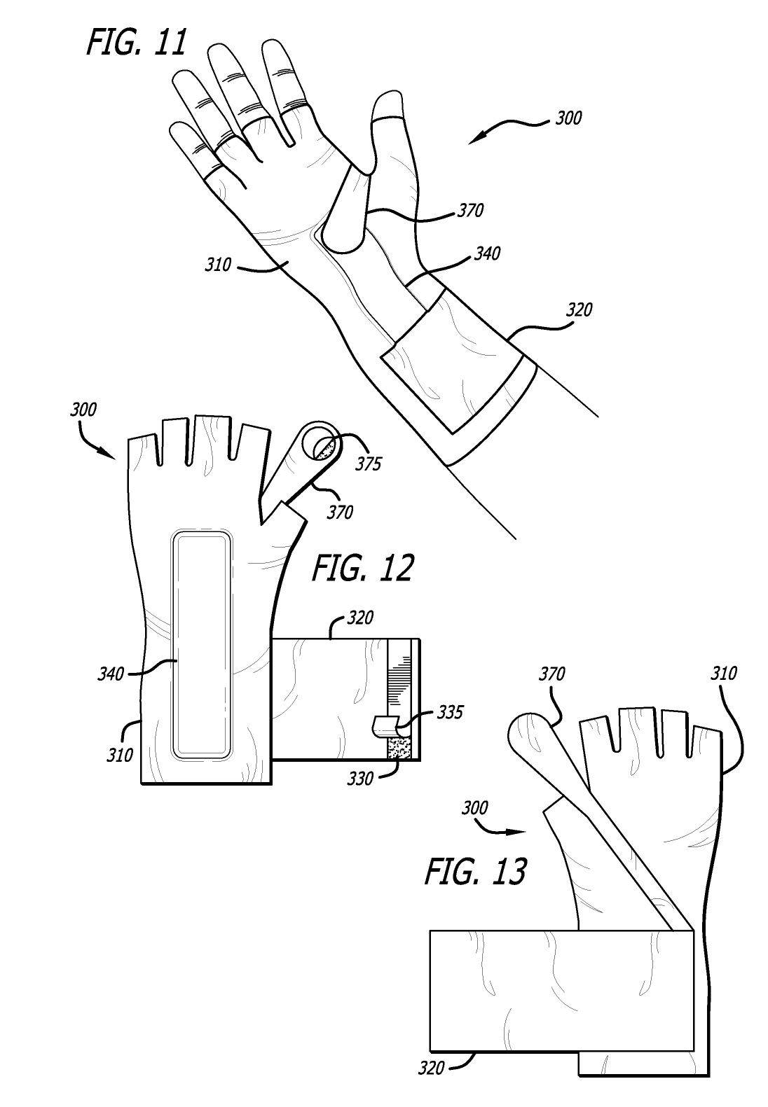

FIGS. 11, 12, and 13 illustrate another disposable wrist support 300 that may be worn under or over a disposable protective glove. The disposable wrist support 300 includes a body 310, which may be a fingerless glove as shown, and a support device such as a wrist strap 320 and/or a support member 340. The support member 340 may be an inner support member disposed to support an inner side of a wearer's wrist, as shown in FIG. 11, or an outer support member disposed to support an outer side of the wearer's wrist (not shown). The support member 340 may be disposed in a sealed pocket formed on the body 310, as shown in FIG. 12, or may be a removable member disposed within an open pocket (similar to the open pocket 260 shown in FIG. 8) in the body 310.

The disposable wrist support 300 may also include a hand strap 370. The handstrap 370 may be configured to wrap around a portion of a wearer's hand, as shown in FIG. 11, to hold an upper end of the support member 340 against the wearer's hand. The hand strap 370 may have an adhesive region 375 that can be exposed by removing a protective liner. The wearer may then affix the end of the hand strap 370 to the body 310 to provide a desired amount of tension, as shown in FIG. 11.

The body 310, the wrist strap 320, and the hand strap 370 may be made from a thin, flexible, stretchable, impervious elastomer material as previously described. The weight or thickness of the material may be selected to provide a desired combination of flexibility, support, and protection. The thickness of the material may be sufficiently thin to allow the disposable wrist support to be worn under conventional protective gloves. The material type and thickness of the body 310, the wrist strap 320, and the hand strap 370 may be the same or different. The body 310 may be provided with or without various inside surface treatments including aloe or powder.

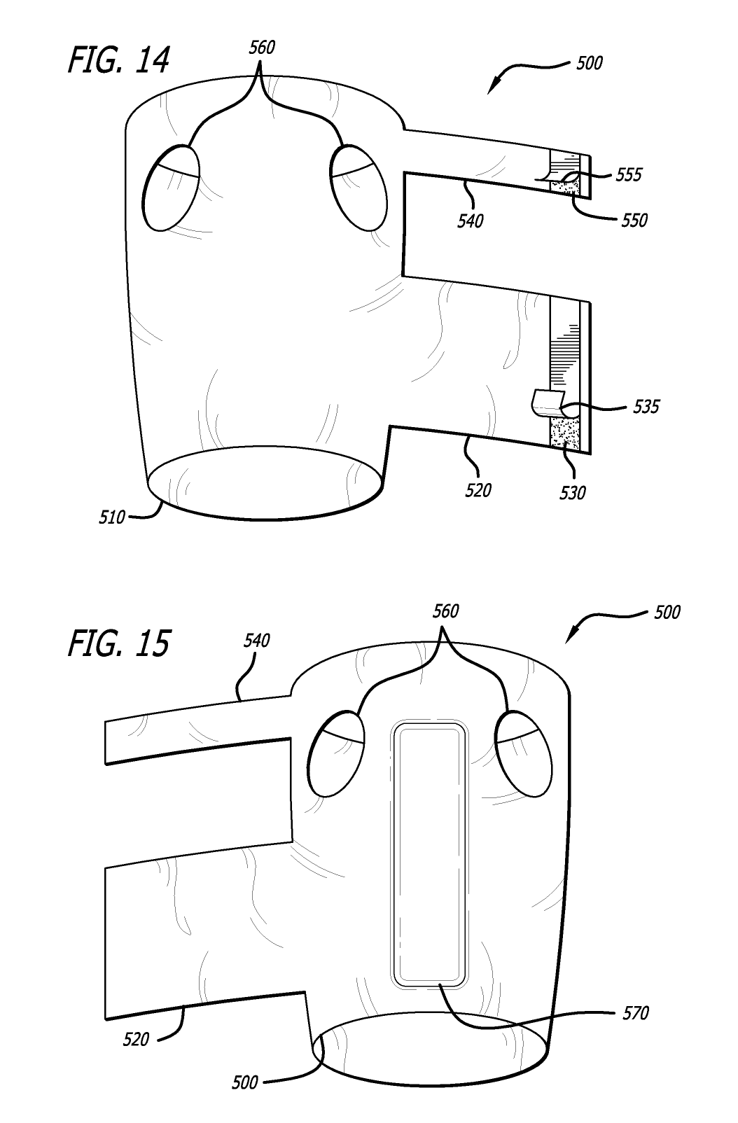

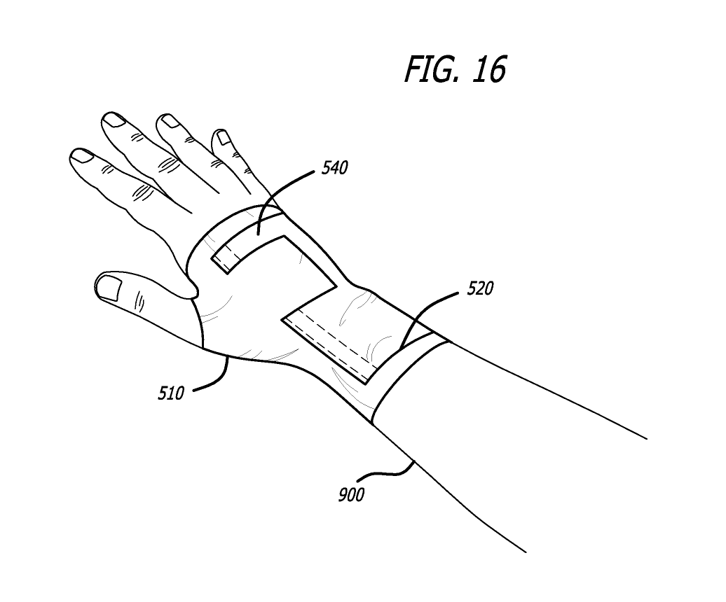

FIGS. 14, 15, and 16 illustrate an ambidextrous disposable wrist support 500 that can be worn over or under a conventional disposable glove. The disposable wrist support 500 may include a body 510 in the form of a tubular sleeve and a support device in the form of one or more of a wrist strap 520, a hand strap 540, and a support member 570. The support member 570 may be sealed within a pocket formed on the body 510 as shown, or may be removable and disposed within an open pocket on the body 510. If the support member is removable, alternative support members may be provided as previously described. The wrist strap 520 and the hand strap 540 may have respective adhesive regions 530, 550 that can be exposed by removing respective protective liners 535, 555. As shown in FIG. 16, the wrist strap 520 and the hand strap 540 may be wrapped about the wrist and hand respectively and fastened to the body 510 to provide a desired amount of compression. The wrist strap 520 and the hand strap 540 may have respective adhesive regions 530, 550 and protective liners 535, 555 on both a front surface and a back surface, allowing the wrist strap and the hand strap to be wrapped about a wearer's wrist in either direction.

The body 510 may have one, two or four thumb holes 560. A wearer may place the disposable wrist support 500 on either the left or right wrist by extending their thumb through the proper thumb hole. If multiple thumb holes are provided, the wearer may place the disposable wrist support 500 on either the left or right wrist with the support member 570 on either the top of the bottom of the wrist by extending their thumb through the proper thumb hole.

The body 510, the wrist strap 520, and the hand strap 540 may be made from a thin, flexible, stretchable elastomer material as previously described. The weight or thickness of the material may be selected to provide a desired combination of flexibility, support, and protection. The thickness of the material may be sufficiently thin to allow the disposable wrist support to be worn under conventional protective gloves. The material type and thickness of the body 510, the wrist strap 520, and the hand strap 540 may be the same or different.

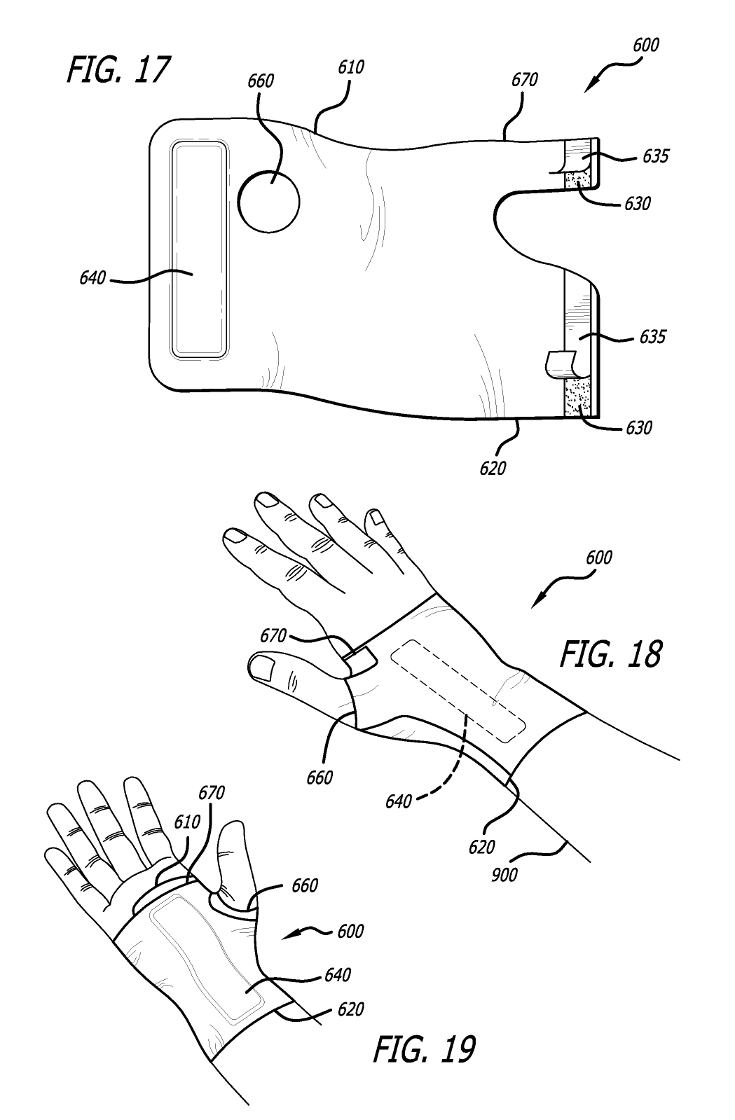

FIGS. 17, 18, and 19 show another disposable wrist support 600 that can be worn over or under a conventional disposable glove. The disposable wrist support 600 may include a body 610, one or more pockets to hold a support member 640, a wrist strap 620 and optionally a hand strap 630. The support member 640 may be disposed in a sealed pocket formed on the body 610, as shown in FIG. 17, or may be a removable member disposed within an open pocket (similar to the open pocket 260 shown in FIG. 8) in the body 610. The wrist strap 620 and the hand strap 670 may have one or more adhesive regions 630 covered by protective liners 635. The wrist strap 620 and the hand strap 670 may have adhesive regions 630 and protective liners 635 on both a front surface and a back surface, allowing the wrist strap and the hand strap to be wrapped about a wearer's wrist in either direction.

The body 610 may have one, two or four thumb holes 660. A wearer may place the disposable wrist support 600 on either the left or right wrist by extending their thumb through the proper thumb hole. The wearer may then wrap the disposable wrist support 600 about their wrist and fasten it in place using the adhesive regions 630. FIG. 18 and FIG. 19 show the disposable wrist support 600 worn on a wearer's wrist with the support member 640 proximate the inner side of the wearer's wrist. The disposable wrist support 600 may be worn on the wearer's wrist with the support member proximate the outer side of the wearer's wrist. As shown, the wrist strap is wrapped about the wearer's wrist above the wearer's thumb and the hand strap is wrapped about the wear's hand below the wearer's thumb.

The body 610 the wrist strap 620 and the hand strap 670 may be made from a single sheet of thin, flexible, stretchable elastomer material as previously described. The weight or thickness of the material may be selected to provide a desired combination of flexibility, support, and protection. The thickness of the material may be sufficiently thin to allow the disposable wrist support to be worn under conventional protective gloves.

Closing Comments

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. With regard to flowcharts, additional and fewer steps may be taken, and the steps as shown may be combined or further refined to achieve the methods described herein. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

1. A disposable wrist support comprising:
a body formed of a flexible impervious elastomer material and configured to be worn about a wearer's wrist; and
at least one support device attached to the body, the at least one support device configured to provide support to the wearer's wrist.

2. The disposable wrist support of claim 1, wherein the body is a glove.

3. The disposable wrist support of claim 1, wherein the body is a sleeve having one or more thumb holes.

4. The disposable wrist support of claim 1, wherein the body comprises:
a sheet of the elastomer material having one or more thumb holes, the sheet configured to wrap about a wearer's wrist; and one or more adhesive regions configured to hold the disposable wrist support in position about the wearer's wrist.

5. The disposable wrist support of claim 4, wherein the one or more adhesive regions include adhesive regions on both an upper surface and a lower surface of the sheet of elastomer material.

6. The disposable wrist support of claim 1, where the at least one support device comprises a wrist strap configured to wrap about a wearer's wrist and fasten with one or more adhesive regions.

7. The disposable wrist support of claim 1, wherein the at least one support device comprises a support member sealed within a closed pocket on the body.

8. The disposable wrist support of claim 1, wherein the at least one support device comprises a support member disposed in an open pocket on the body.

9. The disposable wrist support of claim 8 wherein the support member is selected from a group of interchangeable support members.

10. The disposable wrist support of claim 1, further comprising a hand strap configured to wrap about a wearer's hand and fasten with one or more adhesive regions.

11. The disposable wrist support of claim 1, wherein the support device provides support to the wearer's wrist by resisting motion of the wearer's wrist.

12. The disposable wrist support of claim 1, wherein the support device provides support to the wearer's wrist by limiting a range of motion of the wearer's wrist.

13. The disposable wrist support of claim 1 wherein the disposable wrist support is sufficiently thin to enable a wearer to wear the wrist support underneath a glove of a similar elastomer material.
